(19) United States
(12) Patent Application Publication
(10) Pub. No.: US 2026/0021023 A1
(43) Pub. Date: Jan. 22, 2026

(54) OIL-BASED SOLID COSMETIC

(71) Applicant: SHISEIDO COMPANY, LTD., Tokyo (JP)

(72) Inventors: Akari MIZUGUCHI, Tokyo (JP); Takahiro Katori, Tokyo (JP)

(73) Assignee: SHISEIDO COMPANY, LTD., Tokyo (JP)

(21) Appl. No.: 19/259,407

(22) Filed: Jul. 3, 2025

(30) Foreign Application Priority Data
Jul. 18, 2024 (JP) 2024-114856

(51) Int. Cl. A61K 8/02; A61K 8/891; A61K 8/92; A61Q 17/04

(52) U.S. Cl. CPC A61K 8/0216; A61K 8/891; A61K 8/92; A61Q 17/04

(57) ABSTRACT

provided is an oil-based solid cosmetic for sunscreen use that exhibits excellent water resistance. An oil-based solid cosmetic including a gelling agent (A), a silicone-based surfactant (B) that functions as a water resistance improving agent, and an ultraviolet scattering agent (C). Here, a ratio B/C of the content of the component (B) to the content of the component (C) contained in the oil-based solid cosmetic is 0.01 to 0.5.

### BACKGROUND OF THE INVENTION

### Field of the Invention

The present invention relates to an oil-based solid cosmetic.

### Background Art

Various sunscreen compositions have been developed to minimize the adverse effects of ultraviolet rays on the skin. Generally, sunscreen compositions contain ultraviolet absorbers and ultraviolet scattering agents, which block the penetration of UVA and UVB into the skin and thereby achieve ultraviolet protection effects. Such sunscreen compositions contain ultraviolet absorbers or ultraviolet scattering agents; however, conventionally, emulsified compositions containing an ultraviolet absorber or an ultraviolet scattering agent have been commonly used.

On the other hand, sunscreen compositions are often used under conditions where the user sweats or comes into contact with water. Accordingly, it is required that the composition applied to the skin maintain its ultraviolet protection function without being removed, even when exposed to sweat or water. In addition, there is a demand for non-chemical type sunscreen compositions that do not contain ultraviolet absorbers and aim to achieve sufficient sunscreen effects by means of ultraviolet scattering agents, in order to suppress irritation to the skin.

Conventionally, lotion-type and cream-type sunscreen compositions have been widely used; however, stick-type sunscreen compositions, which do not soil the hands upon application and exhibit high water resistance after application, have also been developed. However, there has been a demand for non-chemical type sunscreen cosmetics with further improved water resistance.

### CITATION LIST

### Patent Literature

Patent Literature 1: JP 2006-52155 A

### SUMMARY OF THE INVENTION

According to the present invention, the following invention is provided.

[1 ] An oil-based solid cosmetic including
a gelling agent (A),
a silicone-based surfactant (B), and
an ultraviolet scattering agent (C), in which
a ratio B/C of a content of the component (B) to a content of the component (C) contained in the oil-based solid cosmetic is 0.01 to 0.5.

[2] The oil-based solid cosmetic according to [1], in which, with respect to a total mass of the oil-based solid cosmetic, a content of the component (A) is 3 to 30 mass %,
a content of the component (B) is 0.2 to 20 mass %, and
a content of the component (C) is 5 to 40 mass %. [3] The oil-based solid cosmetic according to [1] or [2], in which the component (A) is selected from the group consisting of a solid wax and an oil gelling agent.

[4] The oil-based solid cosmetic according to any one of [1] to [3], in which the component (A) is a solid wax.

[5] The oil-based solid cosmetic according to any one of [1] to [4], in which the component (A) is a solid wax having a melting point of 50° C. or higher at normal pressure.

[6] The oil-based solid cosmetic according to any one of [1] to [5], in which the component (B) is a compound in which a polyoxyalkylene chain is bonded to a main chain having a polysiloxane structure.

[7] The oil-based solid cosmetic according to any one of [1] to [6], in which the component (B) has an HLB value of from 0.1 to 18.

[8] The oil-based solid cosmetic according to any one of [1] to [7], in which the component (C) is a particulate metal oxide.

[9] The oil-based solid cosmetic according to any one of [1] to [8], in which the component (C) is hydrophobically treated on its surface.

[10] The oil-based solid cosmetic according to any one of [1] to [9], in which a content of water contained in the oil-based solid cosmetic is 2 mass % or less, based on a total mass of the oil-based solid cosmetic.

[11] The oil-based solid cosmetic according to any one of [1] to [10], further including an oil component (D).

[12] The oil-based solid cosmetic according to any one of [1] to [11], which is a non-emulsified oil-based solid cosmetic.

[13] The oil-based solid cosmetic according to any one of [1] to [12], in which a hardness measured by a rheometer at 37° C. under conditions of a pressure-sensitive shaft of 1.50 φ, a penetration speed of 2 cm/min, and a penetration depth of 10 mm is 20 to 300 mN.

[14] A method for producing an oil-based solid cosmetic,
the method including blending a silicone-based surfactant as a water resistance improving agent into an oil-based solid cosmetic.

[15] An oil-based solid cosmetic including a silicone-based surfactant as a water resistance improving agent.

According to the present invention, there is provided an oil-based solid cosmetic having excellent water resistance and an excellent sunscreen effect when applied to skin.

### DETAILED DESCRIPTION OF THE INVENTION

The oil-based solid cosmetic (hereinafter, sometimes referred to simply as a “cosmetic”) according to the present invention includes
a gelling agent (A),
a silicone-based surfactant (B), and
an ultraviolet scattering agent (C). Each of these components and components that can be additionally blended in will be described below.

### Gelling Agent (A)

The cosmetic according to the present invention includes a gelling agent (hereinafter, the component may be referred to as the component (A)). The gelling agent has an action of gelling and solidifying the composition. Examples of such a gelling agent include a solid wax and an oil gelling agent.

The solid wax described above is not particularly limited as long as it is one that is commonly used in cosmetics. Examples thereof include synthetic waxes such as paraffin wax, microcrystalline wax, polyethylene wax, and Fischer-Tropsch wax; and natural waxes such as carnauba wax, candelilla wax, ecosoy wax, rice wax, beeswax, wood wax, ozokerite, and ceresin. Among these, those that are solid at normal pressure, particularly those having a melting point of 50° C. or higher, are exemplified. However, the solid wax is not limited to these examples.

Examples of the oil gelling agent include condensation products of benzaldehyde compounds and polyhydric alcohols having a valency of 5 or more, such as dibenzylidene sorbitol, tribenzylidene sorbitol, dibenzylidene xylitol, and p-methoxybenzylidene sorbitol;
metal soaps such as calcium stearate, calcium palmitate, lithium 2-ethylhexanoate, and aluminum 12-hydroxystearate; derivatives of N-acyl amino acids, such as amides, esters, and amine salts including lauroylglutamic acid dibutylamide, lauroylglutamic acid stearylamide, dicapryloyllysine laurylamine salt, dicapryloyllysine lauryl ester, and dicapryloyllysine lauroylphenylalanine laurylamide; dextrin fatty acid esters obtained by acid hydrolysis of starch saturated fatty acid esters having an ester substitution degree of 30% or more; and oligomers obtained by condensing 12-hydroxystearic acid with mixed fatty acids composed of glycerin, behenic acid, and eicosanedioic acid. However, the oil gelling agent is not limited to these examples.

The gelling agent can be used alone or in combination of two or more kinds thereof. In addition, among them, a solid wax is preferably used.

### Silicone-based Surfactant (B)

The cosmetic according to the present invention includes a silicone-based surfactant (hereinafter, the component may be referred to as the component (B)). The silicone-based surfactant has a polysiloxane structure in the main chain, and is not particularly limited, but is modified with, for example, a polyether group or a polyglycerin group. Those having an oxyalkylene chain are preferable.

As the silicone-based surfactant modified with a polyether group, various kinds of silicone-based surfactants are known, but the silicone-based surfactant can be arbitrarily selected and used from those that can be used for cosmetics. In addition, the molecular weight of such an activator is not necessarily limited, but those having a mass average molecular weight of 30,000 or more are preferably used.

As examples of polyether-modified silicones modified with a polyether group, the following may be used:
poly(oxyethylene/oxypropylene) methylpolysiloxane copolymer;
polyoxyethylene methylpolysiloxane copolymer;
silicone chain-branched methylpolysiloxane copolymer;
alkyl chain-branched polyoxyethylene methylpolysiloxane copolymer;
alkyl chain-and silicone chain-branched polyoxyethylene methylpolysiloxane copolymer;
crosslinked polyoxyethylene methylpolysiloxane; and
alkyl group-containing crosslinked polyoxyethylene methylpolysiloxane.

Further, as examples of polyglycerin-modified silicones modified with a polyglycerin group, the following may also be used:
branched polyglycerin-modified silicone;
crosslinked polyglycerin-modified silicone;
alkyl group-containing crosslinked polyglycerin-modified silicone; and
alkyl chain-branched polyglycerin-modified silicone.

More specific examples include PEG-9 polydimethylsiloxyethyl dimethicone,
lauryl PEG-9 polydimethylsiloxyethyl dimethicone,
cetyl PEG/PPG-10/1 dimethicone,
PEG-9 methyl ether dimethicone,
PEG-10 dimethicone,
PEG-3 dimethicone,
PEG/PPG-19/19 dimethicone, and
PEG/PPG-18/18 dimethicone;
   however, the present invention is not limited to these examples.

The HLB value of the component (B) can be arbitrarily selected according to the purpose; however, it is preferably 0.1 to 18 and more preferably 1 to 15. Here, the HLB value can be determined by a Griffin method.

Such a silicone-based surfactant remarkably improves the water resistance of the cosmetic according to the present invention. In general, a surfactant is used for improving dispersibility and dispersion stability of an emulsified cosmetic. Therefore, in the oil-based solid cosmetic having no aqueous phase, it is difficult to obtain an effect such as dispersibility improvement. However, it was unexpected that water resistance was improved by combining such an oil-based cosmetic with a silicone-based surfactant among surfactants, particularly a specific silicone-based surfactant as described above.

Therefore, in the present invention, the silicone-based surfactant is blended in as a water resistance improving agent, and the present invention also includes a method for producing an oil-based solid cosmetic or a method for improving water resistance, in which a silicone-based surfactant is blended into an oil-based solid cosmetic as a water resistance improving agent.

Here, the component (B) functions as a water resistance improving agent rather than a surfactant as described above, but since a component generally called a silicone-based surfactant is used, the component (B) is referred to as a surfactant for convenience.

### Ultraviolet Scattering Agent (C)

The cosmetic according to the present invention includes an ultraviolet scattering agent (hereinafter, the component may be referred to as a component (C)). The component (C) can be arbitrarily selected and used from ultraviolet scattering agents used for general sun care cosmetics and the like. Specific examples thereof include fine particulate metal oxides such as zinc oxide, titanium oxide, iron oxide, cerium oxide, and tungsten oxide.

The component (C) may be either untreated or subjected to various types of hydrophobic surface treatment; however, from the standpoint of cosmetic stability, it is preferable to use a hydrophobically treated one. As surface treatment agents used for hydrophobic treatment, those commonly used in the field of cosmetics may be employed. Examples include silicones such as dimethicone and alkyl-modified silicones; alkoxysilanes such as octyltriethoxysilane; dextrin fatty acid esters such as palmitic acid dextrin; and fatty acids such as stearic acid.

The average primary particle size of the component (C) is not particularly limited, but is generally 10 nm to 100 nm, and preferably 10 nm to 50 nm. Here, the average primary particle size in the present specification means the diameter of the primary particles of the powder measured by a generally used method, and specifically, is a value obtained as an arithmetic average of the length of the major axis and the length of the minor axis of the particles from a transmission electron micrograph.

The particle shape of the component (C) is not particularly limited, and is, for example, a spherical shape, an elliptical shape, a crushed shape, or the like.

The cosmetic according to the present invention contains the above-described components (A) to (C) as essential components. The content of each component can be adjusted according to the purpose. For example, with respect to the total mass of the oil-based solid cosmetic,
the content of the component (A) is preferably 3 to 30 mass %, and more preferably 5 to 20 mass %;
the content of the component (B) is preferably 0.2 to 20 mass %, and more preferably 0.5 to 10 mass %; and
the content of the component (C) is preferably 5 to 40 mass %, and more preferably 10 to 30 mass %.

In addition, in the cosmetic according to the present invention, the blending ratio between the component (B) and the component (C) is important. Specifically, the ratio B/C of the content of the component (B) to the content of the component (C) needs to be 0.01 to 0.5, and is preferably 0.03 to 0.3. When B/C is out of this range, the water resistance improving effect tends to be significantly poor.

### Other Components

The cosmetic according to the present invention may contain other components other than the above components (A) to (C) as necessary. As one of such components, an oil component (hereinafter, the component may be referred to as the component (D)) can be contained. The components (D) are those commonly used in cosmetics. Among such oil components, an oil component that is liquid at normal temperature (25° C.) is preferably used in the present invention.

As such component (D), examples include:
ester oils such as dicaprylyl neopentyl glycol, diisostearyl malate, pentaerythrityl tetraethylhexanoate, lauroyl glutamic acid di(phytosteryl/octyldodecyl), triisostearin, glyceryl diisostearate, glyceryl tri(caprylate/caprate), polyglyceryl-6 polyricinoleate, triethylhexanoin, dimer dilinoleic acid (phytosteryl/behenyl), dimer dilinoleic acid (phytosteryl/isostearyl/cetyl/stearyl/behenyl), isopropyl palmitate, triisostearin, ethylhexyl palmitate, myristyl myristate, isopropyl myristate, dipivaloyl tripropylene glycol, diisopropyl sebacate, isodecyl neopentanoate, isocetyl isostearate, cetyl 2-ethylhexanoate, 2-heptylundecyl palmitate, diisobutyl adipate, 2-hexyldecyl sebacate, isopropyl myristate, 2-octyldodecyl oleate, neopentyl glycol diheptanoate, ethylene glycol di-2-ethylhexanoate, tri-2-heptylundecanoic acid glyceride, glyceryl diisostearate, glyceryl triisostearate, glyceryl trioctanoate, glyceryl triisopalmitate, trimethylolpropane tri-2-ethylhexanoate, or pentaerythritol tetra-2-ethylhexanoate;
hydrocarbon oils such as squalane;
linear silicone oils such as dimethicone, dimethylpolysiloxane, or methylphenylpolysiloxane (diphenylsiloxyphenyltrimethicone);
cyclic silicone oils such as octamethylcyclotetrasiloxane or decamethylcyclopentasiloxane;
higher alcohols, fatty acids, triglycerin, and fluorine-modified oils.

In the case where the component (D) is blended in the cosmetic of the present invention, a content of the component (D) based on a total mass of the cosmetic can be arbitrarily adjusted depending on purposes and the like; however, it is preferable that the content of the component (D) is 80 mass % or less, and more preferably 70 mass % or less. The component (D) according to the present invention does not include the component (A) or an ultraviolet absorber described later.

The cosmetic according to the present invention achieves a protective effect against ultraviolet rays by the above-described component (C); however an ultraviolet absorber may be combined in order to further improve the effect. Examples of the ultraviolet absorber include benzoic acid derivatives, salicylic acid derivatives, cinnamic acid derivatives, dibenzoylmethane derivatives, β, β-diphenylacrylate derivatives, benzophenone derivatives, benzylidene camphor derivatives, phenylbenzimidazole derivatives, triazine derivatives, phenylbenzotriazole derivatives, anthranilate derivatives, imidazoline derivatives, benzalmalonate derivatives, 4,4-diarylbutadiene derivatives, and silicone derivatives.

However, since the ultraviolet absorber may cause stickiness when applied, it is preferable that the content thereof is low, and more preferably that it is not contained at all.

In addition, the cosmetic according to the invention can also contain water. Water may be blended as a solvent contained in various raw materials. However, the cosmetic of the present invention preferably has a low water content in order to maintain the solid shape. Specifically, it is preferable that a content of water contained in the cosmetic is 2 mass % or less based on a total mass of the cosmetic, and more preferably, the water is not contained at all. The cosmetic according to the present invention is an oil-based solid cosmetic having a low water content, and is preferably a non-emulsified solid cosmetic. Since the content of water is low, it is considered that the component (B) hardly exhibits a function as a surfactant that is usually exhibited, such as improvement of dispersibility stability. However, in the present invention, the component (B) exhibits a function as a water resistance improving agent at the time of application in a non-emulsified solid cosmetic.

The cosmetic according to the present invention may optionally contain other optional components. Examples of such components include
a surfactant (a) (other than the component (B), for example, anionic surfactants, nonionic surfactants, cationic surfactants, and amphoteric surfactants);
a moisturizer (b) (for example, 1,3-butylene glycol, propylene glycol, dipropylene glycol, glycerin, diglycerin, and the like);
a powder component (c) (other than the component (C), for example, inorganic powders, organic powders, pigments, and the like);
a lipophilic thickener (d);
a lower alcohol (e) (alcohols having less than 6 carbon atoms);
an antioxidant (f) (for example, tocopherols, dibutylhydroxytoluene (BHT), and the like);
a preservative (g) (for example, methylparaben, phenoxyethanol, and the like);
an anti-inflammatory agent (h) (for example, glycyrrhizic acid derivatives, glycyrrhetinic acid derivatives, and the like);
a whitening agent (i) (for example, saxifrage extract, arbutin, tranexamic acid, and the like);
various extracts (j);
an activator (for example, royal jelly and the like);
a blood circulation promoter (k) (for example, nonanoic acid vanillylamide, benzyl nicotinate, and the like);
an anti-seborrheic agents (I) (for example, sulfur, thiantol, and the like);
an anti-inflammatory agent (m) (for example, tranexamic acid, thiotaurine, hypotaurine, and the like); and
a film-forming agent (n).

Since the cosmetic according to the present invention has a solid shape, it can have, for example, a stick shape. It is preferable that the cosmetic according to the present invention has a relatively high hardness in order to directly apply the stick-shaped cosmetic to the skin or the like. Specifically, it is preferable that the hardness measured by a rheometer at 37° C. under conditions of a pressure-sensitive shaft of 1.5 φ, a penetration speed of 2 cm/min, and a penetration depth of 10 mm is 20 to 300 mN, and more preferably 30 to 200 mN.

For the measurement, for example, a rheometer manufactured by Sun Scientific Co., Ltd. can be used.

### EXAMPLES

The present invention will be specifically described based on the following examples, but the present invention is not limited to these examples. The content is represented in mass % with respect to the total amount, unless otherwise specified.

### Examples 1 to 10, Comparative Examples 1 to 6

Oil-based solid cosmetics of examples and comparative examples were prepared with the formulation shown in Table 1.

Furthermore, regarding the performance of these cosmetics, water resistance was evaluated as follows.

### Evaluation of Water Resistance

A measurement sample in an amount of 2 mg/cm² was applied to a plate (S plate) (V-groove PMMA plate of 5 cm×5 cm, SPFMASTER-PA01, manufactured by Fuji Kasei Co., Ltd.) with a finger for 60 seconds and dried for 15 minutes. Next, the absorption spectrum at 280 nm to 500 nm of the plate to which the measurement sample was added was measured with a spectrophotometer (U-3500 self-recording spectrophotometer, manufactured by Hitachi, Ltd.). Thereafter, the measured plate was immersed in a water tank filled with water having a hardness of 50 to 500, and the water in the water tank was stirred at 300 rpm for 30 minutes using a three-one motor. Thereafter, the plate was taken out from the water tank, allowed to grow for about 15 minutes to about 30 minutes at room temperature (25° C.±5° C.) until water droplets on the surface of the plate disappeared, and the light absorption spectrum was measured again. From the absorbance (Abs) integrated values at 280 nm to 500 nm before and after water bath, the rate of change in absorbance (Abs) before and after water bath was calculated based on Formula (1) below. The higher the value of the absorbance change rate before and after water bath, the more the measurement sample does not flow out into the water bath, so that it can be said that the water resistance is high. Therefore, the absorbance change rate before and after water bath was evaluated as “water resistance”.

### Formula (1)

Absorbance change rate before and after water bath (%)=(integrated absorbance value after water bath)/(integrated absorbance value before water bath)×100

The obtained results are as shown in Table 1.

TABLE 1

| | | Examples | | | | | | | |
|---|---|---|---|---|---|---|---|---|---|
| | | 1 | 2 | 3 | 4 | 5 | 6 | 7 | 8 |
| | [symbol] thylene/Microcrystalline wax*1 | 9 | 9 | 9 | 9 | 9 | 9 | 9 | 9 |
| | [symbol] 10 Dimethicone (HLB = 2) | 1 | 2 | 3 | 5 | 1 | 2 | 2 | 2 |
| C | [symbol] ic acid-treated titanium | 5 | 5 | 5 | 5 | 5 | 5 | 5 | 5 |
| | dioxide fine particles*2 | | | | | | | | |
| | [symbol] ne-treated zinc oxide particles*3 | 18 | 18 | 18 | 18 | 18 | 18 | 18 | 18 |
| | [symbol] xide microparticles-treated | | | | | | | | |
| | with dextrin palmitate*4 | | | | | | | | |
| D | [symbol] thicone | 10 | 10 | 10 | 10 | 10 | | | |
| | [symbol] nylsiloxyphenyl trimethicone | 19.98 | 18.98 | 17.98 | 15.98 | 18.98 | 19.98 | 9.98 | |
| | [symbol] entyl glycol dicaprate | 20 | 20 | 20 | 20 | 20 | 29 | 39 | 48.98 |
| | [symbol] tearyl malate | 17 | 17 | 17 | 17 | 17 | 17 | 17 | 17 |
| a | [symbol] ydroxystearic acid | | | | | 1 | | | |
| | [symbol] tan sesquiisostearate | | | | | | | | |
| | [symbol] lyceryl-6 polyricinoleate | | | | | | | | |
| f | [symbol] herols | 0.02 | 0.02 | 0.02 | 0.02 | 0.02 | 0.02 | 0.02 | 0.02 |
| | [symbol] | 0.0015 | 0.0015 | 0.0015 | 0.0015 | 0.0015 | 0.0015 | 0.0015 | 0.0015 |
| | Total | 100 | 100 | 100 | 100 | 100 | 100 | 100 | 100 |
| B/C | | 0.0435 | 0.087 | 0.13 | 0.217 | 0.0435 | 0.087 | 0.087 | 0.087 |
| Water resistance | | 91 | 97 | 95 | 96 | 88 | 82 | 81 | 85 |
| | | Examples | | Comparative Examples | | | | | |
| | | 9 | 10 | 1 | 2 | 3 | 4 | 5 | 6 |
| | [symbol] thylene/Microcrystalline wax*1 | 9 | 9 | 9 | 9 | 9 | 9 | | 9 |
| | [symbol] 10 Dimethicone (HLB = 2) | 2 | 2 | | | | | 2 | 14 |
| C | [symbol] ic acid-treated titanium | 5 | 5 | 5 | 5 | 5 | 5 | 5 | 5 |
| | dioxide fine particles*2 | | | | | | | | |
| | [symbol] ne-treated zinc oxide particles*3 | 18 | | 18 | 18 | 18 | 18 | 18 | 18 |
| | [symbol] xide microparticles-treated | | 18 | | | | | | |
| | with dextrin palmitate*4 | | | | | | | | |
| D | [symbol] thicone | 10 | 10 | 10 | 10 | 10 | 10 | 10 | 10 |
| | [symbol] nylsiloxyphenyl trimethicone | | 18.98 | 20.98 | 18.98 | 18.98 | 18.98 | 18.98 | 6.98 |
| | [symbol] entyl glycol dicaprate | 39.98 | 20 | 20 | 20 | 20 | 20 | 29 | 20 |
| | [symbol] tearyl malate | 17 | 17 | 17 | 17 | 17 | 17 | 17 | 17 |
| a | [symbol] ydroxystearic acid | | | | 2 | | | | |
| | [symbol] tan sesquiisostearate | | | | | 2 | | | |
| | [symbol] lyceryl-6 polyricinoleate | | | | | | 2 | | |
| f | Tocopherols | 0.02 | 0.02 | 0.02 | 0.02 | 0.02 | 0.02 | 0.02 | 0.02 |
| | BHT | 0.0015 | 0.0015 | 0.0015 | 0.0015 | 0.0015 | 0.0015 | 0.0015 | 0.0015 |
| | Total | 100 | 100 | 100 | 100 | 100 | 100 | 100 | 100 |

| | | | | | | | | |
|---|---|---|---|---|---|---|---|---|
| B/C | 0.087 | 0.087 | 0 | 0 | 0 | 0 | 0.087 | 0.609 |
| Water resistance | 97 | 75 | 56 | 37 | 59 | 71 | 61 | 54 |

*1Mixture of polyethylene (85 mass %) and microcrystalline wax (15 mass %). Melting point 82° C. to 90° C.
*2Particle size 10 to 50 μm
*3Particle size 10 to 60 μm
*4Particle size 10 to 50 μm [symbol]
indicates data missing or illegible when filed

### Examples 11 to 18

Oil-based solid cosmetics of examples in which the type of the component (B) was changed with the formulation shown in Table 2 were prepared. The obtained cosmetic was evaluated for water resistance in the same manner as in Example 1. The obtained results are as shown in Table 2.

TABLE 2

| | | Examples | | | | | | | |
|---|---|---|---|---|---|---|---|---|---|
| | | 11 | 12 | 13 | 14 | 15 | 16 | 17 | 18 |
| | [symbol] ethylene/Microcrystalline wax*1 | 9 | 9 | 9 | 9 | 9 | 9 | 9 | 9 |
| B | [symbol] -12 Dimethicone (HLB = 8) | 2 | | | | | | | |
| | [symbol] -9 Polydimethylsiloxyethyl | | 2 | | | | | | |
| | dimethicone (HLB = 3.8) | | | | | | | | |
| | [symbol] l PEG/PPG-10/1 dimethicone (HLB = 5) | | | 2 | | | | | |
| | [symbol] yl PEG-9 polydimethylsiloxyethyl | | | | 2 | | | | |
| | dimethicone (HLB = 2.8) | | | | | | | | |
| | [symbol] utyl dimethicone polyglyceryl-3 | | | | | 2 | | | |
| | (HLB = 0.48) | | | | | | | | |
| | [symbol] -10 Dimethicone/Tocopherol (HLB = 2) | | | | | | 2 | | |
| | [symbol] -10 Methyl ether dimethicone (HLB = 14) | | | | | | | 2 | |
| | [symbol] -11 methyl ether dimethicone (HLB = 14) | | | | | | | | 2 |
| C | [symbol] ric acid-treated titanium | 3 | 3 | 3 | 3 | 3 | 3 | 3 | 3 |
| | dioxide fine particles*2 | | | | | | | | |
| | [symbol] ne-treated zinc oxide particles*3 | 18 | 18 | 18 | 18 | 18 | 18 | 18 | 18 |
| D | [symbol] thicone | 10 | 10 | 10 | 10 | 10 | 10 | 10 | 10 |
| | [symbol] enylsiloxyphenyl trimethicone | 20.98 | 20.98 | 20.98 | 20.98 | 20.98 | 20.98 | 20.98 | 20.98 |
| | [symbol] entyl glycol dicaprate | 20 | 20 | 20 | 20 | 20 | 20 | 20 | 20 |
| | [symbol] stearyl malate | 17 | 17 | 17 | 17 | 17 | 17 | 17 | 17 |
| f | Tocopherols | 0.02 | 0.02 | 0.02 | 0.02 | 0.02 | 0.02 | 0.02 | 0.02 |
| | BHT | 0.0015 | 0.0015 | 0.0015 | 0.0015 | 0.0015 | 0.0015 | 0.0015 | 0.0015 |
| | Total | 100 | 100 | 100 | 100 | 100 | 100 | 100 | 100 |

| | | | | | | | | |
|---|---|---|---|---|---|---|---|---|
| B/C | 0.10 | 0.10 | 0.10 | 0.10 | 0.10 | 0.10 | 0.10 | 0.10 |
| Water resistance | 84 | 93 | 96 | 97 | 79 | 92 | 87 | 90 |

*1Mixture of polyethylene (85 mass %) and microcrystalline wax (15 mass %). Melting point 82° C. to 90° C.
*2Particle size 10 to 50 μm
*3Particle size 10 to 60 μm [symbol]
indicates data missing or illegible when filed

1. An oil-based solid cosmetic comprising:
a gelling agent (A);
a silicone-based surfactant (B); and
an ultraviolet scattering agent (C), wherein
a ratio B/C of a content of the component (B) to a content of the component (C) contained in said oil-based solid cosmetic is 0.01 to 0.5.

2. The oil-based solid cosmetic according to claim 1, wherein, with respect to a total mass of said oil-based solid cosmetic,
a content of the component (A) is 3 to 30 mass %,
a content of the component (B) is 0.2 to 20 mass %, and
a content of the component (C) is 5 to 40 mass %.

3. The oil-based solid cosmetic according to claim 1, wherein the component (A) is selected from the group consisting of a solid wax and an oil gelling agent.

4. The oil-based solid cosmetic according to claim 1, wherein the component (A) is a solid wax.

5. The oil-based solid cosmetic according to claim 1, wherein the component (A) is a solid wax having a melting point of 50° C. or higher at normal pressure.

6. The oil-based solid cosmetic according to claim 1, wherein the component (B) is a compound in which a polyoxyalkylene chain is bonded to a main chain having a polysiloxane structure.

7. The oil-based solid cosmetic according to claim 1, wherein the component (B) has an HLB value of 0.1 to 18.

8. The oil-based solid cosmetic according to claim 1, wherein the component (C) is a particulate metal oxide.

9. The oil-based solid cosmetic according to claim 1, wherein the component (C) is hydrophobically treated on its surface.

10. The oil-based solid cosmetic according to claim 1, wherein a content of water contained in said oil-based solid cosmetic is 2 mass % or less, based on a total mass of said oil-based solid cosmetic.

11. The oil-based solid cosmetic according to claim 1, further comprising an oil component (D).

12. The oil-based solid cosmetic according to claim 1, which is a non-emulsified oil-based solid cosmetic.

13. The oil-based solid cosmetic according to claim 1, wherein a hardness measured by a rheometer at 37° C. under conditions of a pressure-sensitive shaft of 1.5 φ, a penetration speed of 2 cm/min, and a penetration depth of 10 mm is 20 to 300 mN.

14. A method for producing an oil-based solid cosmetic, the method comprising blending a silicone-based surfactant as a water resistance improving agent into an oil-based solid cosmetic.

15. An oil-based solid cosmetic comprising a silicone-based surfactant as a water resistance improving agent.
